(19) United States
(12) Patent Application Publication
(10) Pub. No.: US 2017/0209614 A1
(43) Pub. Date: Jul. 27, 2017

(54) SCENTED ORIGAMI VEHICLE AND ROOM FRESHENERS

(71) Applicant: Jaime Ramos, Moorpark, CA (US)

(72) Inventor: Jaime Ramos, Moorpark, CA (US)

(21) Appl. No.: 15/008,244

(22) Filed: Jan. 27, 2016

(51) Int. Cl. A61L 9/12

(52) U.S. Cl. CPC A61L 9/12

(57) ABSTRACT

One or more flat substrate sheets are provided with folding lines and slits, tabs, and apertures. The sheet or sheets are folded in origami style to create a three-dimensional object. The substrate sheet or sheets include fragrant material so as to emit a pleasant fragrance.

### RELATED APPLICATION

This application claims priority to U.S. Provisional application No. 62/108,598 filed Jan. 28, 2015.

### FIELD OF INVENTION

The present invention generally relates to air fresheners. More particularly, the present invention relates to sheets of scented material which are stamped, cut, or perforated, and folded in origami style to create a three-dimensional air freshener.

### BACKGROUND OF THE INVENTION

Air fresheners are commonly used in automobiles. Typically, such air fresheners comprise a two-dimensional sheet of material which has been impregnated with a scent and in some cases dye-stamped into a basic geometric shape, such as a tree or the like. Such air fresheners are typically hung by means of a looped string over a door handle, rearview mirror of the automobile, or the like to provide the desired scent within the car and mask undesirable odors. Such air fresheners are relatively inexpensive to manufacture and purchase. Moreover, these air fresheners are relatively easy to transport and store for the distributor and retailer. However, such air fresheners are uninspiring and dull.

There exist other air fresheners, used both in the automobile and in the home or office setting, which are three-dimensional in shape and more elaborate. Such air fresheners typically include a reservoir of scented oil, paste, or the like which is slowly emitted over time. In some cases, the air freshener requires a source of electricity in order to operate. However, such air fresheners are much more expensive as the manufacturing thereof is more complicated and they take up additional space and weight when transporting from the manufacturer and when stored by the retailer.

Accordingly, there is a continuing need for an air freshener which can be used in the automobile, home, or office which is inexpensive to manufacture, transport and store, and which provides an interesting three-dimensional object when in use. The present invention fulfills these needs and provides other related advantages.

### SUMMARY OF THE INVENTION

The scented origami vehicle and room fresheners of the present invention comprise, generally, a flat origami body having a plurality of folding lines on a flat substrate sheet, and a fragrant substance embedded, infused or applied to the substrate sheet. The substrate sheet may be erected into a three-dimensional origami air freshener through bending of the substrate sheet along the folding lines.

In one of embodiment, the flat substrate sheet includes a slot or aperture for attaching the three-dimensional origami air freshener to another object such as the rear view mirror of an automobile. The slot or aperture accommodates a rear view mirror support post.

Means are also provided for protecting the rear view mirror from contact with the fragrant substance. The fragrant substance preferably includes natural or organic scents. Such natural or organic scents may comprise Lavender, jasmine, Chamomile, or Peppermint.

In another embodiment, the origami air freshener may include a plurality of a flat substrate sheets each having at least one folding line, which are each erected and then assembled to one another to form the three-dimensional origami air freshener.

The flat substrate sheet may be provided with two free ends attached to one another and which is pre-folded along one of the folding lines such that at least two flat portions of the substrate sheet lie against one another prior to being erected into the 3-dimensional origami air freshener. Preferably, the flat substrate sheet is pre-tensioned such that it tends to self-erect into its three-dimensional configuration.

Other features and advantages of the present invention will become apparent from the following more detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a top view of a pre-fabricated air freshener shown in sheet form, embodying the present invention;

FIG. 2 is a perspective view of the sheet of FIG. 1 folded to create a three-dimensional air freshener;

FIG. 3 is a perspective view of the air freshener of FIG. 2 attached to a rearview mirror of an automobile;

FIG. 4 illustrates a plurality of flat, unfolded air freshener sheets similar to FIG. 1, showing exemplary alternative configurations;

FIG. 5 is a perspective view of a three-dimensional air freshener, in the form of a dog, created from the flat, unfolded sheets of FIG. 4;

FIG. 6 is a front elevational view of the air freshener of FIG. 5;

FIG. 7 is a rear elevational view of the air freshener of FIG. 5;

FIG. 8 is a left side and partially sectioned view of the air freshener of FIG. 5;

FIG. 9 is a top plan view of the air freshener of FIG. 5;

FIG. 10 illustrates another embodiment of a scented origami air freshener that involves a single scented substrate sheet folded flat and which can be erected with a simple pulling motion at each end, as illustrated by the arrows;

FIG. 11 is the fully erected configuration of the origami air freshener of FIG. 10;

FIG. 12 illustrates yet another exemplary embodiment where a scented origami air freshener may be pre-formed and shipped in a collapsed or flattened configuration, and then erected with a simple pulling motion as shown by the arrow; and

FIG. 13 illustrates the fully erected configuration of the scented origami air freshener of FIG. 12.

### DETAILED DESCRIPTION OF THE PREFERRED EMBODIMENTS

As shown in the accompanying drawings for purposes of illustration, the present invention resides in a scented origami air freshener. It is to be understood at the outset of the description that persons of skill in the appropriate arts may modify the invention herein described while still achieving the favorable results of this invention.

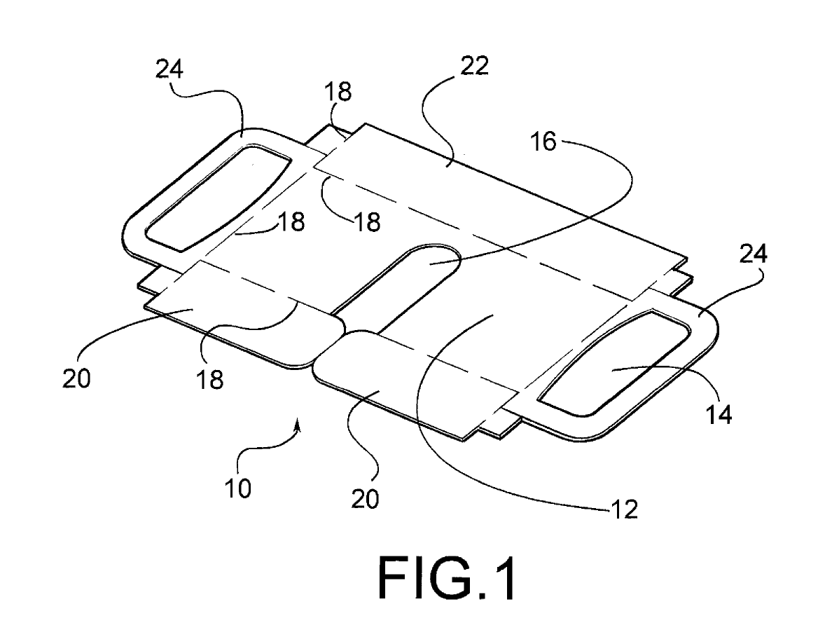

With reference to FIG. 1, an air freshener 10 embodying the present invention in an unfolded and flat state is shown. The air freshener 10 is comprised of one or more sheets of material 12 (or substrate sheets 12) which may include cardboard, wood, plastic, foam, paper, or other light construction materials which are capable of being folded manually into the desired shape. As such, the sheet of material 12 comprising the air freshener 10 is of a flexible substrate configured to be folded into an origami.

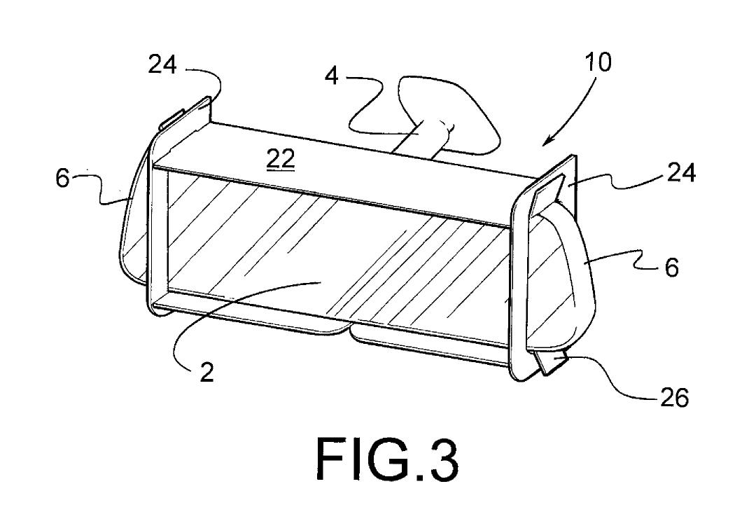

The sheet of material 12 is typically die cut so as to include the necessary apertures, slits or openings, and the like to create the desired shape and potential attachment points to other objects. The air freshener 10 of FIG. 1 is configured, upon folding, to be fit around a rearview mirror 2, as illustrated in FIG. 3. As such, the air freshener 10 includes slots or apertures 14 designed to have portions of the rearview mirror 2, such as the ends 6 thereof inserted therethrough, and other slots or apertures 16, such as for insertion of the air freshener 10 over a supporting post 4 of the rearview mirror 2. It is contemplated by the present invention that the apertures, slots, etc., 14 and 16 be formed by perforations which will enable the easy removal of the material from the sheet 12 to create the necessary aperture 14, slit or slot 16.

The sheet of material 12 also includes a plurality of folding or score lines formed thereon. The folding lines 18 may comprise printed folding lines or a structure integrated into the sheet of material 12 and configured to facilitate substantially precise folding of the sheet 12 along the structures to be generated (e.g., score lines). Moreover, the folding lines may be formed, for example, by means of partial perforations or the like which extend either partially into the sheet of material 12, or which extend fully through the sheet of material 12, but which result in the folding and creation of a line in the sheet of material 12 when the portions thereof are folded, but do not detach from one another.

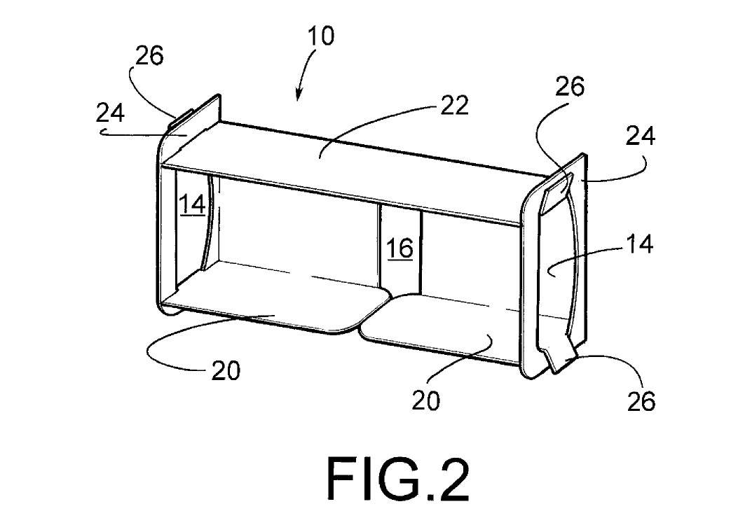

With reference now to FIG. 2, an assembled three-dimensional air freshener 10 is illustrated wherein portions of the sheet of material 12 have been folded according to the folding lines 18 provided on the sheet of material 12. In this case, two pivoting flaps 20 are created which enable the air freshener 10 to be disposed over the rearview mirror post 4 such that the post 4 resides within slot 16. An upper shelf 22 will rest upon a top surface of the rearview mirror, as illustrated in FIG. 3. Opposing ends 6 of the rearview mirror extend through apertures 14 formed in the side walls 24 of the air freshener 10. Tabs 26 may be bent downwardly or upwardly, such as from the pivoting flaps 20 and the shelf 22 over the side walls 24 so as to hold the various members of the air freshener 10 in place.

With reference now to FIG. 3, the air freshener 10 is shown mounted upon an automobile's rearview mirror 2. The air freshener 10 may be pre-colored, pre-printed, etc., with a desirable color scheme, graphics, or even advertising material. The air freshener 10 will be easily viewable from within the car by the driver and passengers. In fact, every time that the driver looks into the rearview mirror 2, the driver will see the air freshener 10 mounted thereon, which can be advantageously used for advertising purposes. Other benefits of the air freshener 10 include the creation of a three-dimensional object which enables air freshener scent to be more easily distributed within the automobile.

It will be appreciated that the air freshener 10 illustrated in FIGS. 1-3 attaches to the rearview mirror 2 without any adhesives or fasteners, and can be easily placed on and removed from the mirror 2 as needed and desired by merely removing tabs from corresponding slots or unbending the tabs 26 so as to disassemble the air freshener 10, which can later be reassembled.

A fragrant material may be formed on the sheet of substrate material 12 or the sheet may be embedded with a fragrant material or infused with such fragrant material such that upon removing the sheet of material 12 from an enclosure, such as a plastic bag (not shown), fragrance will be emitted from the air freshener 10. Such fragrant material may be in the form of a liquid into which the sheet of material 12 is placed so that the sheet of material 12 is soaked or otherwise impregnated in the fragrant material, or sprayed onto the substrate sheet of material 12, or injected into or formed as a layer of the sheet of material 12. Additionally, a layer of fragrant material may be placed onto the substrate sheet 12 and covered, such as with a peel-away covering, such that the peel away covering essentially blocks the emission of the fragrance from the fragrant material until the peel away cover is removed so as to expose the fragrant material on the sheet 12.

The origami air freshener 10 of the present invention may employ synthetic, natural and/or organic scents such as Lavender, jasmine, Chamomile, Peppermint, and the like, for therapeutic relief. Advantageously, such scents promote well-being of the automobile occupant. Moreover, such origami air fresheners 10 may be sold with a kit that includes fragrant refills.

On some designs, the automobile interior such as the windshield and/or the rear view mirror 2 may need to be protected from fragrant residue which is deem harmful to glass and plastic. In such cases, the origami air fresheners 10 would be provided with coatings (not shown) to contain the fragrant oils and to prevent any contact with the automobile interior components.

In an alternative configuration, secondary objects supported by the substrate sheet are the vehicle and engine for the fragrance. Such secondary objects, for example, could comprise raised objects which incorporate or are comprised of the fragrant material and could comprise a portion of the design of the air freshener 10. For example, such objects could comprise eyes of an animal figure, the eyes being filled with a liquid or a gel fragrant material. It will be appreciated that the entire substrate sheet 12 may infused or covered with fragrant material, or only portions thereof.

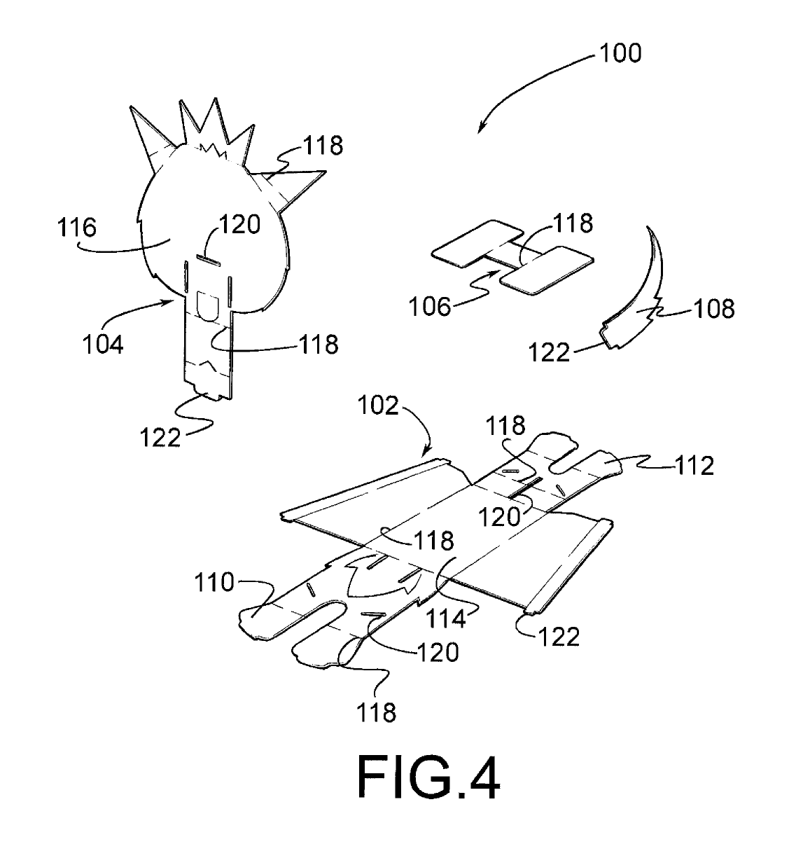

With reference now to FIGS. 4-9, yet another air freshener 100 embodying the present invention is shown. In this case, multiple substrate sheets 102-108 are provided. These may be pre-cut into the individual pieces or included as a single sheet and removed from one another, such as by perforations or the like. The substrate sheets 102-108 correspond to portions of the final three-dimensional object to be created which are independently folded and created and attached to one or more of the other sheet members 102-108.

These sheets of substrate 102-108 are similar to that described above and may be comprised of a variety of materials, including cardboard, styrene or other plastics, paper, foam, or other light construction materials. One or more of the substrate sheets 102-108 will be of a material which enables the user to manually fold the substrate sheets 102-108 in a manner to create a three-dimensional object.

With continuing reference to FIG. 4, the various substrate sheets 102-108 are cut into a configuration and design which, either in and of itself or after folding, creates the desired three-dimensional object or a portion thereof. For example, the substrate sheet portion 108 comprises a tail of the three-dimensional dog, as illustrated in FIG. 5. Substrate sheet 102 is cut and configured to form a body portion of the three-dimensional dog air freshener. This includes front feet 110 and rear feet 112 of the dog, and a body portion 114 thereof. Substrate sheet 104 comprises a head 116 portion of the final three-dimensional dog air freshener. Sheet or portion 106 serves as an attachment between the head 116 and the body 114 of the air freshener.

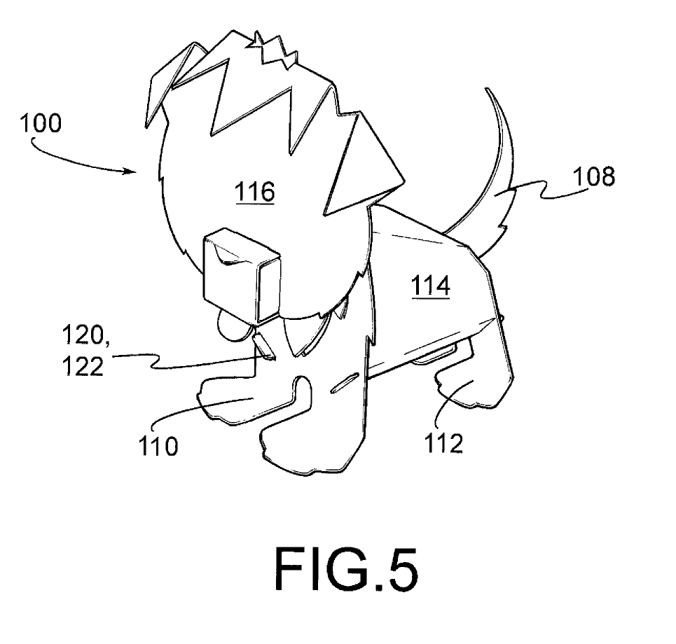

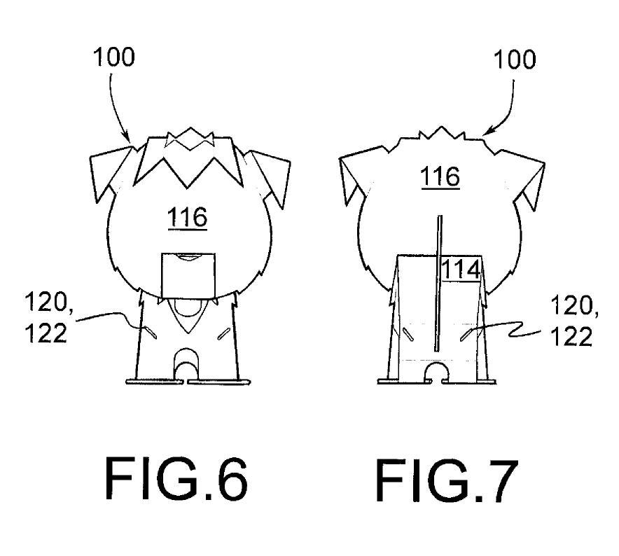

It will be seen that there are a plurality of folding lines 118 which are either printed upon or integrally formed by perforations or the like, in the various substrate sheets 102-108 to indicate where the user should manually fold the material, and in some cases to facilitate the folding thereof. Slits 120 are formed in strategic locations in various substrate sheets 102-108 for insertion of tabs 122 of various portions of the various sheets 102-108 for attaching portions of the sheets to other sheets or components to create the final three-dimensional object, as illustrated in FIG. 5 where it can be seen that a three-dimensional dog structure is created as the final air freshener 100 upon folding and assembling the various pieces illustrated in FIG. 4 to one another. FIG. 6 illustrates a front view of the dog air freshener 100, and FIG. 7 illustrates a rear view of the dog air freshener 100.

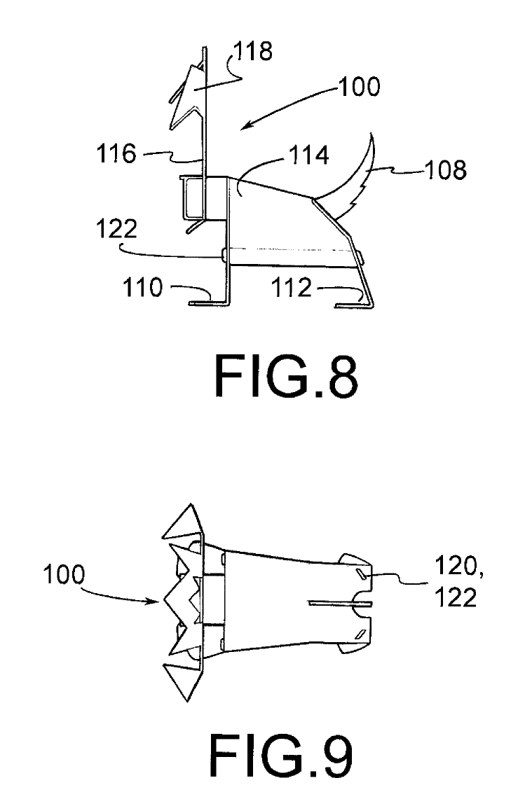

FIG. 8 is a side view, which is partially sectioned, illustrating interconnection of the various component parts after they have been folded to one another in order to create the three-dimensional dog structure of the air freshener 100. This includes the insertion of tabs 122 through corresponding slits 120.

FIG. 9 is a top plan view of the three-dimensional dog configured air freshener 100, illustrating various connecting points of tabs and slits and folds necessary to create the structure.

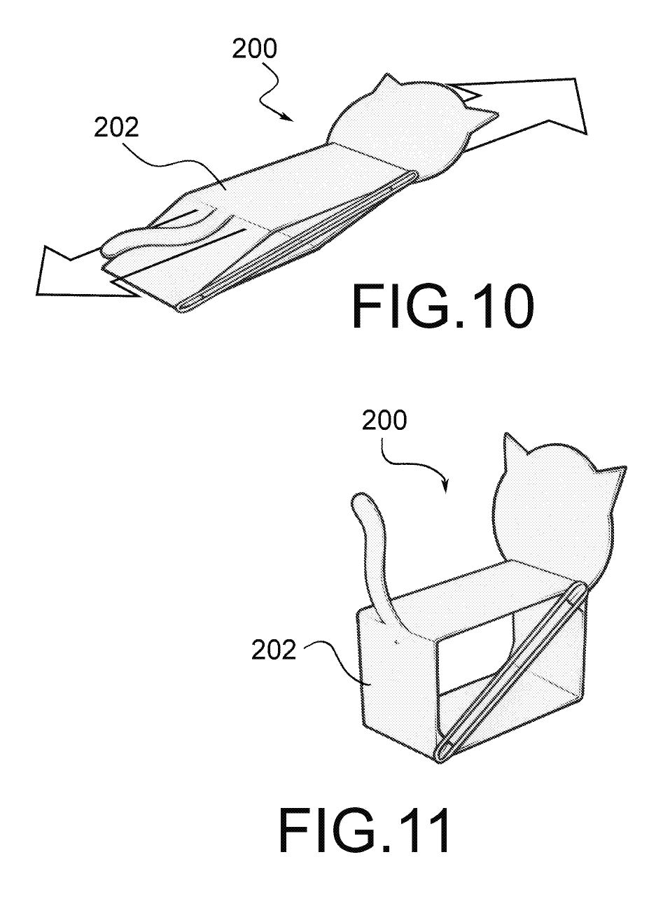

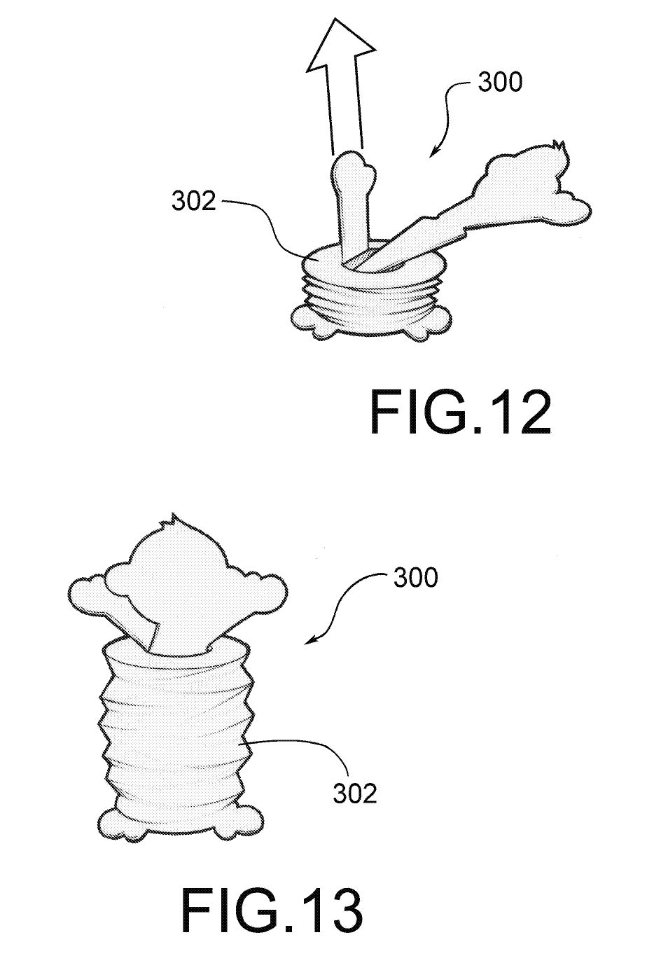

FIGS. 10-13 show additional types of scented origami air fresheners 200, 300 constructed in accordance with the principles of the present invention. These drawings show origami air freshener models that may be erected with a simple motion, either through rubber bands, notches and/or material tension. Removable tape strips could be utilized to erect an air freshener removing the tape to release tension on a flat packaged origami air freshener. In other words, the substrate sheets 202, 302 utilized could be configured to have a built in tension when shipped in the flattened configurations (FIGS. 10 and 12) such that when removed from packaging there is a tendency for them to self-erect as shown in FIGS. 11 and 13.

In this regard, FIG. 10 illustrates another embodiment of a scented origami air freshener that involves a simple scented substrate sheet folded flat and which can be erected with a simple pulling motion at each end, as illustrated by the arrows FIG. 11 is the fully erected configuration of the origami air origami air freshener of FIG. 10. FIG. 12 illustrates yet another exemplary embodiment where a scented origami air freshener may be pre-formed and shipped in a collapsed or flattened configuration, and then erected with a simple pulling motion as shown by the arrow. Moreover, FIG. 13 illustrates the fully erected configuration of the scented origami air freshener of FIG. 12.

From the foregoing, it will be appreciated that there are a wide variety of three-dimensional objects which can be formed into a three-dimensional air freshener in accordance with the present invention, and either attached to existing objects, such as a rearview mirror, or which are capable of standing and being supported on their own. Users will enjoy creating the air fresheners, and the air fresheners will be much more visually appealing and interesting than others on the market. Moreover, such air fresheners made in accordance with the present invention are easily manufactured, shipped, and stored.

Although several embodiments have been described in detail for purposes of illustration, various modifications may be made without departing from the scope and spirit of the invention. Accordingly, the invention is not to be limited, except as by the appended claims.

What is claimed is:

1. An origami air freshener, comprising:
a flat origami body having a plurality of folding lines on a flat substrate sheet; and
a fragrant substance embedded, infused or applied to the substrate sheet;
whereby the substrate sheet may be erected into a three-dimensional origami air freshener through bending of the substrate sheet along the folding lines.

2. The origami air freshener of claim 1, including a plurality of flat substrate sheets each having at least one folding line, wherein the substrate sheets are each erected and then assembled to one another to form the three-dimensional origami air freshener.

3. The origami air freshener of claim 1, wherein the flat substrate sheet has two ends attached to one another and is pre-folded along one of the folding lines such that at least two flat portions of the substrate sheet lie adjacent to one another prior to being erected into the three-dimensional origami air freshener.

4. The origami air freshener of claim 3, wherein the flat substrate sheet is pre-tensioned such that it tends to self-erect into its three-dimensional configuration.

5. The origami air freshener of claim 1, wherein the flat substrate sheet includes a slot or aperture for attaching the three-dimensional origami air freshener to another object.

6. The origami air freshener of claim 5, wherein the three-dimensional origami air freshener is attached to a rear view mirror of an automobile, and wherein the slot or aperture accommodates a rear view mirror support post.

7. The origami air freshener of claim 6, including means for protecting the rear view mirror from contact with the fragrant substance.

8. The origami air freshener of claim 1, wherein the fragrant substance includes synthetic, natural or organic scents.

9. The origami air freshener of claim 8, wherein the scents comprise Lavender, jasmine, Chamomile, or Peppermint.

10. An origami air freshener, comprising:
a flat origami body having a plurality of folding lines on a flat substrate sheet, wherein the flat substrate sheet includes a slot or aperture for attaching the three-dimensional origami air freshener to another object;
a fragrant substance embedded, infused or applied to the substrate sheet, wherein the fragrant substance includes natural or organic scents;
whereby the substrate sheet may be erected into a three-dimensional origami air freshener through bending of the substrate sheet along the folding lines.

11. The origami air freshener of claim 10, wherein the three-dimensional origami air freshener is attached to a rear view mirror of an automobile, and wherein the slot or aperture accommodates a rear view mirror support post, and including means for protecting the rear view mirror from contact with the fragrant substance.

12. The origami air freshener of claim 11, wherein the natural or organic scents comprise Lavender, jasmine, Chamomile, or Peppermint.

13. The origami air freshener of claim 10, including a plurality of flat substrate sheets each having at least one folding line, wherein the substrate sheets are each erected and then assembled to one another to form the three-dimensional origami air freshener.

14. The origami air freshener of claim 10, wherein the flat substrate sheet has two ends attached to one another and is pre-folded along one of the folding lines such that at least two flat portions of the substrate sheet lie adjacent to one another prior to being erected into the three-dimensional air freshener.

15. The origami air freshener of claim 14, wherein the flat substrate sheet is pre-tensioned such that it tends to self-erect into its three-dimensional configuration.

16. An origami air freshener, comprising:
a flat origami body having a plurality of folding lines on a flat substrate sheet, wherein the flat substrate sheet includes a slot or aperture for attaching the three-dimensional origami air freshener to another object, wherein the flat substrate sheet has two ends attached to one another and is pre-folded along one of the folding lines such that at least two flat portions of the substrate sheet lie adjacent to one another prior to being erected into a three-dimensional origami air freshener through bending of the substrate sheet along the folding lines, and wherein the flat substrate sheet is pre-tensioned such that it tends to self-erect into its three-dimensional configuration; and
a fragrant substance embedded, infused or applied to the substrate sheet.

17. The origami air freshener of claim 16, wherein the three-dimensional origami air freshener is attached to a rear view mirror of an automobile, and wherein the slot or aperture accommodates a rear view mirror support post, and including means for protecting the rear view mirror from contact with the fragrant substance.

18. The origami air freshener of claim 16, wherein the fragrant substance includes natural or organic scents comprising Lavender, Jasmine, Chamomile or Peppermint.

19. The origami air freshener of claim 16, including a plurality of flat substrate sheets each having at least one folding line, wherein the substrate sheets are each erected and then assembled to one another to form the three-dimensional origami air freshener.
